(19) United States
(12) Patent Application Publication
(10) Pub. No.: US 2018/0303976 A1
(43) Pub. Date: Oct. 25, 2018

(54) COVALENT MODIFICATION OF DECELLULARIZED ALLOGENEIC GRAFTS WITH ACTIVE PHARMACEUTICALS

(71) Applicant: THOMAS JEFFERSON UNIVERSITY, PHILADELPHIA, PA (US)

(72) Inventors: Noreen J. Hickok, Philadelphia, PA (US); Constantinos Ketonis, Philadelphia, PA (US); Javad Parvizi, Gladwyne, PA (US); Irving M. Shapiro, Philadelphia, PA (US); Christopher S. Adams, Lansdowne, PA (US); John A. Abraham, Philadelphia, PA (US)

(73) Assignee: THOMAS JEFFERSON UNIVERSITY, PHILADELPHIA, PA (US)

(21) Appl. No.: 15/765,309

(22) PCT Filed: Oct. 3, 2016
(86) PCT No.: PCT/US16/55151

§ 371 Date: Apr. 2, 2018

Related U.S. Application Data
(60) Provisional application No. 62/236,617, filed on Oct. 2, 2015.

(51) Int. Cl. A61L 27/54; A61L 27/36

(52) U.S. Cl. CPC A61L 27/54; A61L 27/365; A61L 2430/02; A61L 2300/406; A61L 27/3608

(57) ABSTRACT

A functionalized tissue having covalently bound antibiotics and methods for producing the functionalized tissue having bound antibiotics comprising; providing a tissue having a sufficient number of primary amine groups on the tissue; coupling and deprotecting one or more F-moc AEEA linkers using HATU chemistry to the primary amine groups; and coupling an antibiotic to said AEEA linkers using HATU chemistry.

### CROSS-REFERENCE TO RELATED APPLICATIONS

The present application claims priority to U.S. Provisional Patent Application No. 62/236,617, filed Oct. 2, 2015, which application is incorporated herein by reference in its entirety.

### GOVERNMENT SUPPORT

This invention was made with government support under DE019901, HD061053 and AR051303, awarded by the National Institutes of Health. The government has certain rights in the invention.

### FIELD OF INVENTION

The present application is generally related to devices and methods thereof to prevent infections through the use of biologically enhanced allografts.

### BACKGROUND OF INVENTION

Infection is a problem that besets all implants. After the first year of loading, 10-50% of failures for oral implants are caused by peri-implantitis (24; 25); in external orthopaedic fixation, infection rates vary from 0.6-45.2% (26-30). Implants and supplemental bone material used for joint or spinal surgeries show a <1% and ˜4% infection rate; those used as structural adducts after trauma or cancer can reach infection rates of 40%. When the implant is designed to interface with the tissue, surfaces are designed to foster cell adhesion. Unfortunately, fostering cellular attachment also fosters bacterial colonization as both processes rely on the same ECM proteins. Infection risks increase when patients are immunocompromised by conditions like cancer, diabetes (31), osteoporosis (32) or aging (33).

Titanium or other metal alloys are frequently employed in these implant materials. Unfortunately the Ti alloy implant itself is often readily colonized by bacteria (34; 35). Similarly, the supplemental allograft bone that can be used for bone augmentation or as a structural graft after trauma or excision of a tumor is an ideal surface for bacterial colonization. Once bacteria are adherent, they become encased in a fibrous matrix and biofilm slime that limits antibiotic efficacy and immune surveillance. As the biofilm colony matures, bacteria bud off the surface to seed the surrounding tissues as well as the implant. Studies have shown that this insensitivity to antibiotics occurs rapidly and that antibiotic levels many times the minimum inhibitory concentration (MIC) are unable to kill adherent bacteria. Thus, if a biofilm is established on an implant, removal of the prosthesis and infected tissue followed by aggressive antibiotic/antiseptic treatments may be the only successful therapy.

Traditionally, treatment of implant infection relies on local antibiotic delivery systems including methylmethacrylate cement (36-39), biodegradable polymers (40-43) and, more recently, nanotubes (44). However, these antibiotic cement spacers have multiple limitations (45-49) and may, on occasion, fail to eradicate infection. Furthermore, they are inappropriate for application in a soft tissue environment. Antibiotics immobilized in a biodegradable polymer, commonly poly-lactic acid (PLA), poly-glycolic acid (PGA), or a combination of both (PLAGA) (50-54), can be used as a nail, plate or as a coating for a prosthetic surface. The intrinsic dissolution rate of the polymer determines the kinetics of antibiotic release (55-58). These, and other biodegradable systems such as sol-gel films and hydrogels (56; 59-66) can release bioactive molecules at the site of infection. However, the drawbacks include the indiscriminate release of antibiotics, their inherent fragility, and once depleted of antibiotic, the carrier itself, whether polymer or cement, can serve as a site for bacterial colonization (67-69). Although these approaches are extremely powerful, they cannot deliver long-term protection from infection.

In some circumstances, it is more advantageous to use bone or tissue materials instead of metal materials. Approximately 1.5 million bone and tissue allografts are distributed each year by American Association of Tissue Banks-accredited tissue banks in the United States.¹ Grafting of this allograft has become a vital part of orthopedic surgery, and it is estimated that the use of bone allograft is required for more than 800,000 musculoskeletal procedures performed annually in the United States alone.^{2, 3} Today bone graft is the second most often transplanted tissue^{3, 4} exceeded only by blood. Implantation of synthetic or processed tissue or Biomaterials into the human body is plagued by bacterial infection.⁵⁻⁸ Once bacteria adhere to a nonliving surface, they proliferate and, in a permissive environment, secrete a complex polymeric biofilm that protects the embedded bacteria from immune surveillance and greatly attenuates antibiotic effectiveness.⁹

Bacterial colonization and biofilm formation is particularly prevalent when bone allografts are utilized as structural adducts. These grafting materials are highly porous, noncellular, and avascular, and are thus inaccessible to immune surveillance, local cellular defense mechanisms,¹⁰ and systemic antibiotics. More than 11% of implanted bone grafts develop infection¹¹⁻¹⁴ necessitating reoperations, removal of foreign material, debridement, aggressive lavage and lengthy antibiotic treatment. To combat infection, grafts of synthetic materials that release high local concentrations of antibiotics from coatings or controlled release systems have been created.^{3, 15, 16} With bone in particular, direct adsorption of antibiotics to allograft is used as an elution system.¹⁷ These elution systems have met with varying degrees of success, and their use is limited by concerns over development of resistance, and ultimately re-establishment of infection as antibiotic elution wanes.

In summary, allograft bone is frequently used throughout the world for bone supplementation in areas where bone autograft is either not available or not sufficient. Because these large grafts may become infected, especially when used in subjects who have experienced severe trauma or are immunocompromised because of different chemotherapies, finding new methods to prevent graft infection is critical.

### SUMMARY OF INVENTION

Accordingly, embodiments disclosed herein are directed to materials that utilize an amine linkage from the tissue to a linker, which is then connected to a pharmaceutical material, such as an antibiotic material. Such linkage mechanism provides for high levels of pharmaceutical materials at the implant bone surface. Reduction or elimination of the high infection rates could be drastically reduced by creation of an implant that could resist bacterial colonization whether residing within a tissue such as bone or serving as a percutaneous prosthesis and that could remain biocompatible.

A method for manufacturing a bone allograft tissue comprising a covalently linked antibiotic comprising: revealing primary amines through partial demineralization of the bone allograft; coupling followed by deprotection of between one and four F-moc AEEA linkers using HATU chemistry to the primary amines; coupling of antibiotic using either HATU chemistry of a carboxylic acid if available on the antibiotic or succinyl chemistry if only a primary amine or amide is available on the antibiotic; and eluting of the unbound antibiotic leaving antibiotic covalently bound to tissue.

A functionalized bone material comprising at least one linker covalently bonded to an amine groups on the surface of the functionalized bone material, wherein a pharmaceutical composition is further bonded to the linker.

A method for manufacturing a tissue comprising a covalently linked antibiotic comprising: revealing a sufficient number of primary amine on the surface of the tissue; coupling followed by deprotection of between one and four F-moc AEEA linkers using HATU chemistry to the primary amines; coupling of a suitable antibiotic to said linkers; and eluting of the unbound antibiotic leaving antibiotic covalently bound to the linker.

A method for manufacturing a bone allograft tissue comprising a covalently linked antibiotic comprising: hydrating the bone material; demineralizing the hydrated bone material to reveal primary amine groups on the surface of the bone material; coupling and deprotecting one or more F-moc AEEA linkers using HATU chemistry to the primary amine groups; and coupling an antibiotic to said AEEA linkers using HATU chemistry. In certain embodiments, the method may further comprise chemically protecting the first antibiotic; coupling and deprotecting one or more F-moc AEEA linkers using HATU chemistry; coupling a second antibiotic to said AEEA linkers using HATU chemistry; and eluting the unbound second antibiotic from the bone.

A tissue having a sufficient number of primary amine on the surface of the material, wherein attached to the primary amine is a linker group and attached to the opposing end of the linker group is an antibiotic.

A demineralized bone product having on the surface of the bone a sufficient number of primary amine and attached to a portion of the primary amine are between 1-4 linkers and attached to the opposing end of the linker a covalently bonded antibiotic.

In a further embodiment, a process for creating a sufficient concentration of an antibiotic to a material comprising revealing primary amines on the surface of the material, adding a coupling linker to the primary amines on the surface of the material wherein the other end of the coupling linker is attached to an antibiotic.

A demineralized bone product comprising a sufficient number of antibiotics linked to primary amines on the surface of the bone. In a preferred embodiments, the demineralized bone product is defined by having a sufficient concentration of primary amines so as to provide a sufficient number of connection points for attaching antibiotics to the demineralized bone product. In further embodiments, the demineralized bone product can be defined by having a sufficient concentration of antibiotics. In certain embodiments, this can be defined as having a a ratio of 6 fold more amines than the minimum number of antibiotics needed. In other embodiments, the concentration of antibiotics is defined as 26 ng/mg of bone.

In a further embodiment, a material is defined as having a sufficient number of primary amine disposed on the surface of the material, wherein said primary amine are suitable for attaching to 1-4 linkers, and attached to the opposing end of the linkers is an antibiotic. In certain embodiments, the number of linkers corresponds to the particular antibiotic selected in order to create increased efficacy.

In certain embodiments, the material is defined by a product made by a process identified herein.

In certain embodiments, use of a material having bonded thereto a sufficient number of antibiotics to a linker attached to a primary amine from the material, wherein said material is suitable for reducing infection or presence of bacteria.

In a further embodiment, a method for manufacturing a tissue comprising a covalently linked antibiotic comprising; revealing primary amines through partial demineralization of the allograft; coupling followed by deprotection of between one and four F-moc AEEA linkers using HATU chemistry; coupling of antibiotic using either HATU chemistry of a carboxylic acid if available on the antibiotic or succinyl chemistry if only a primary amine or amide is available on the antibiotic; and eluting of the unbound antibiotic leaving antibiotic covalently bound to tissue.

In a further embodiment, a method for manufacturing a tissue comprising a covalently linked antibiotic comprising; revealing primary amines through partial demineralization of the allograft or using existing amines on an allograft tissue surface, or a combination thereof; coupling followed by deprotection of between one and four F-moc AEEA linkers using HATU chemistry; coupling of antibiotic using either HATU chemistry of a carboxylic acid if available on the antibiotic or succinyl chemistry if only a primary amine or amide is available on the antibiotic; and eluting of the unbound antibiotic leaving antibiotic covalently bound to tissue.

A further embodiment is directed to a method for manufacturing a tissue comprising a covalently linked antibiotic comprising: hydrating a bone material; demineralizing the hydrated bone material to reveal primary amine groups on the surface of the bone material; coupling and deprotecting one or more F-moc AEEA linkers using HATU chemistry; coupling an antibiotic to said AEEA linkers using HATU chemistry; and eluting the unbound antibiotic from the bone.

A further embodiment is directed to a method for manufacturing a tissue comprising a covalently linked antibiotic comprising: hydrating a bone material; demineralizing the hydrated bone material to reveal primary amine groups on the surface of the bone material; coupling and deprotecting one or more F-moc AEEA linkers using HATU chemistry; coupling an antibiotic to said AEEA linkers using succinyl chemistry; and eluting the unbound antibiotic from the bone.

A further embodiment is directed to a functionalized bone material comprising at least one linker covalently bonded to an amine group on the surface of the functionalized bone material, wherein an antibiotic material is further bonded to the linker. A further embodiment comprises wherein the antibiotic material comprises two or more antibiotics selected from the group consisting of VAN, DOX, and tetracycline.

A further embodiment is directed to a method for manufacturing a tissue comprising a covalently linked antibiotic comprising: revealing primary amines through partial demineralization of the allograft or other treatment to achieve a minimum of 26 ng/mg of bone of antibiotic: coupling followed by deprotection of between one and four F-moc AEEA linkers using HATU chemistry; coupling of antibiotic using either HATU chemistry if a carboxylic acid is available on the antibiotic or succinyl chemistry if only a primary amine or amide is available on the antibiotic; and eluting of the unbound antibiotic leaving antibiotic covalently bound to tissue.

A further embodiment is directed to a method for manufacturing a tissue comprising a covalently linked antibiotic comprising: hydrating a bone material; demineralizing the hydrated bone material to reveal primary amine groups on the surface of the bone material; coupling and deprotecting one or more F-moc AEEA linkers using HATU chemistry; coupling an antibiotic to said AEEA linkers using HATU chemistry; chemically protecting the antibiotic and performing a second demineralization process on the bone to reveal additional primary amine groups on the surface of the bone; coupling and deprotecting one or more F-moc AEEA linkers using HATU chemistry; coupling an antibiotic to said AEEA linkers using HATU chemistry; and eluting the unbound antibiotic from the bone. A further embodiment may deprotect the antibiotics before or after the final elution step.

A further embodiment is directed to a method for manufacturing a tissue comprising a covalently linked antibiotic comprising: hydrating a bone material; demineralizing the hydrated bone material to reveal primary amine groups on the surface of the bone material; coupling and deprotecting one or more F-moc AEEA linkers using HATU chemistry; coupling at least two different antibiotics to said AEEA linkers using HATU chemistry; and eluting the unbound antibiotic from the bone.

### BRIEF DESCRIPTION OF THE DRAWINGS

FIGS. 1A and 1B depict the number of free primary amines that increase with initial allograft demineralization. FIG. 1A details the increased exposure of amines, as detected by fluorescamine staining over a 72 hour demineralization. FIG. 1B shows the concentration of Ca²⁺ measured in solution during demineralization of allograft bone. The arrow indicates the 3-day demineralization used for the syntheses described.

FIGS. 2A and 2B depict that demineralization increases the accessibility of primary amines. A. Graph showing the number of amines detected by ninhydrin staining for mineralized allograft, partially demineralized (3 days, 12.5% EDTA), or fully demineralized bone allograft. B. Staining by fluorescamine of the available primary amines in mineralized, partially demineralized, and demineralized bone. Background pictures show the level of the innate fluorescence of bone allograft.

FIG. 3. Reaction scheme for attachment of vancomycin to bone allograft. Allograft is first partially demineralized (steps 1-4), followed by coupling with two (2-(2-amino-ethoxy)-ethoxy)-acetic acid, and finally coupling of antibiotic using chemistry with Fmoc protecting groups.

FIGS. 4A and 4B depict measurement of recovered secondary antibody after specific binding to VAN-allograft. A. A standard curve for fluorescence of the AlexaFlour488 antibody in the 7M urea extraction buffer. B. Based on the standard curve, the amounts of antibody recovered after treatment of antibody-stained control or VAN-allograft bone. Note the background levels of VAN obtained in the control bone due to non-specific binding.

FIGS. 5A-5D depict that VAN-allograft shows decreased S. aureus colonization. Control (a) or different batches of VAN-allograft (b-d) were incubated with S. aureus to test activity.

FIG. 6 depicts a schematic scheme for binding of DOX to allograft bone.

FIGS. 7A-7D depict two tests of Control and DOX-allograft against S. aureus or E. coli.

FIGS. 8A-8F depict bacteria on the bone and images of DOX-allograft on the bone.

FIG. 9 depicts colony counts of S. aureus and E. coli for DOX-allograft or control.

FIGS. 10A and 10B depict Phalloidin staining of hFOBs on DOX-allograft showing normal morphology.

### DETAILED DESCRIPTION

Infection associated with inert implants is complicated by bacterial biofilm formation that renders the infection recalcitrant to antibiotic treatment. The methods and materials described herein describe attaching antibiotic compounds or compositions to a modified bone allograft or other tissue that aids in rendering the tissue inhospitable to bacterial colonization and the establishment of infection.

In view of the need to prevent or reduce bone infection when using metal or metal alloy materials, antibiotics have been bound to metal surfaces using self-assembled monolayers of aminopropyltriethoxy silane.^{18, 19} These covalently modified surfaces resist bacterial colonization in vitro,^{20, 21} and appear to ameliorate the signs of infection in vivo²². However, the chemistries for use with regard to natural materials, including bone allografts and autografts do not necessitate this similar connection mechanism. Therefore, novel strategies were required to determine what materials could potentially bind an antibiotic, and how to bind that antibiotic appropriately to ensure that it remained active on certain tissues.

Allogenic grafts are harvested from human cadavers and extensively used in clinical practice to fill bony defects and serve as structural components for deficits encountered in orthopaedics, dentistry, and medicine. Infection remains one of the most devastating complications associated with bone graft use. However, by covalently bonding antibiotics to morselized allograft bone the allograft is protected from bacterial colonization.

Other tissues that are also used as allografts are harvested and can be utilized for surgical procedures. For example, certain veinous tissues, tendons, ligaments, and cardiac grafts can be utilized as a donor tissue. In certain instances, these materials have a sufficient number of primary amines on the surface of the material and do not need to undergo a demineralization process, as with the bony material, to generate a surface sufficient to allow for attachment of antibiotics as is described through this application. Therefore, where sufficient number of amines are present, these tissues can be utilizes as the donor tissue for purposes of attaching antibiotics to the donor tissue.

The embodiments herein describe a method and materials that covalently link antibiotics directly to extracellular matrix (ECM) proteins of bone and other connective tissues and vascular structures, including but not limited to arteries or veins, to convert a passive allograft into a bioactive surface that resists bacterial colonization, while maintaining biocompatibility, which, heretofore was impossible with such materials.

Particularly described in the embodiments herein are processes, products, and products made by the processes to affix antibiotics to a material having exposed free amines on the surface of the material. For example, there are several known classes of antibiotics that are suitable against Gram positive and Gram negative bacteria. These classes of antibiotics are known to one of ordinary skill in the art. In certain embodiments, it is advantageous to utilize antibiotics in the classes of glycylcyclines, tet family, and glycopeptide antibiotics. These can be advantageously affixed to between 0-4 linking elements (linkers), wherein the opposing end of the linker is attached to the free nitrogen on the product surface.

Research Design and Methods

Several types of material are suitable for generating sufficient free amine on the surface of the material to be utilized in the embodiments described herein. In a preferred embodiment, bone allograft material is utilized to be coated with antibiotics in an effort to provide for a concentrated antibiotic graft material to help reduce or prevent infection once the allograft material is used in a surgical procedure.

The bone allograft material cannot simply be coated with an antibiotic, or adsorb the antibiotic materials, instead the antibiotic needs to be actually attached to the surface of the material to prevent or reduce infection. For a synthesis of an antibiotic-modified bone allograft or tissue, a first challenge was that the allograft lacked sufficient functional linker groups to which to attach any material to the surface. Accordingly, through experimentation, an investigation into the number of primary amines present in the bone matrix was undertaken to allow for effective modification of the surface.

Doxycycline (DOX) or (DOXY), Vancomycin (VAN), tetracycline, and other antibiotics were investigated for attaching to materials as well as for efficacy against certain Gram negative and Gram positive bacterial. With particular regard to attaching DOX, the DOX was utilized in conjunction with two Fmoc-AEEA linkers and attached to bone allograft. DOX is particularly suited for use as an antibiotic in surgical cases as DOX is effective against Gram-negative and some Gram-positive bacteria. Therefore, bone grafts coated with DOX provide a strong tool against polymicrobial infections often found in patients undergoing chemotherapy after resection of large bony tumors.

Preparation of Antibiotics to the Bone:

The availability of surface amines becomes a limiting factor in choosing the proper material for tethering antibiotics. In evaluating bone material, only after modifying the bone structure was there suitable surface amine groups for bonding. Specifically, decellularized bone is comprised of an organic, predominantly collagenous matrix that is impregnated with inorganic, calcium-rich mineral.²⁵ This ECM is an attractive option for attachment since the collagenous component should be rich in amino acids that bear side chains with primary amines. Nevertheless, recent modeling of collagen suggests that the majority of primary amines may be clustered in highly charged pockets in the gap regions of collagen fibrils. These same sites are thought to act as nucleation sites for initiation of mineral deposition.²⁶ Thus, access to this region is necessary for modifying the collagenous ECM. Indeed, demineralization and by implication, exposure of these primary amine-containing pockets significantly increased amine availability, as detected by the Ninhydrin assay and fluorescamine staining.

Therefore, a step to perform a limited partial demineralization resulted in apparently uniform amine exposure on the surface of the allograft with fluorescence intensity akin to that of fully demineralized allograft. Nevertheless, the total amine content of this partially demineralized bone was only moderately greater than fully mineralized bone and significantly lower than fully demineralized bone.

However, the studies provided evidence that the majority of amines must still be entrenched in the bone mineral, and that only a surface demineralization occurred. Importantly, when measuring released Ca²⁺ during this partial demineralization, it appeared that demineralization at the early time points was very effective at exposing the surface amines, while preserving much of the mineral in the core of the bone, thereby preserving most of its structural and mechanical stability. It is worth noting that this result was obtained with a mixture of cortical and cancellous bone and that the appearances were sufficiently similar as to allow no discrimination based on microscopy.

The proteins supply the primary amines for antibiotic bonding include collagen type I. The collagen type I provides for major sites for antibiotic bonding, both because of its abundance in the bone matrix and because of the effect of demineralization on exposure of primary amines. VAN can be advantageously attached where the primary amine allows formation of a peptide bond between it and the carboxylic acid of VAN. Importantly, this peptide bond should not affect VAN activity. From a structural perspective, this carboxylic acid falls outside of the region mapped for peptidoglycan binding and thus should not affect the active site of the antibiotic.

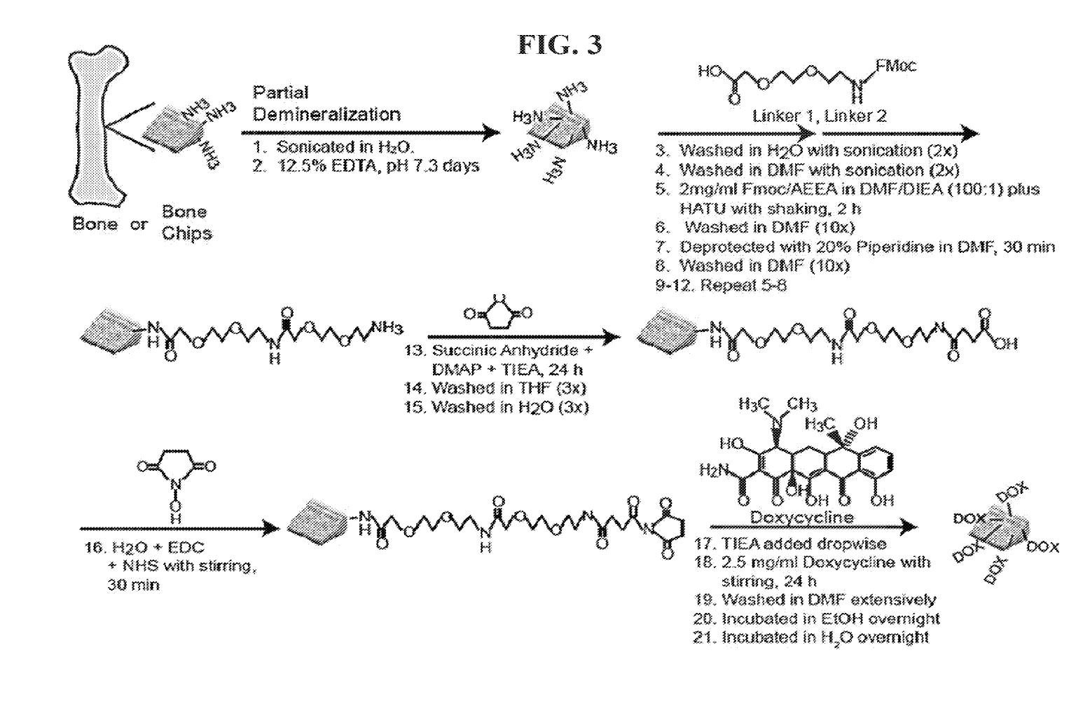

FIG. 3 provides a detail of the process for preparation of the bone material and for coupling the DOX to the bone for subsequent use. Cancellous/cortical human bone granules 0.5-2.0 mm (kind gift of Musculoskeletal Transplant Foundation, MTF) as well as cortical bone stubs cut into 1×1 cm squares using a high-speed cutter (Dremel®), were washed and sonicated with dH2O until washings were clear. The samples were partially demineralized with 12.5% EDTA, pH 7, for 3 days with shaking; EDTA was replaced daily. Prior to synthesis, all samples were washed with dH20 and sonicated twice for 30 min in dimethylformamide (DMF, Argos Organics).

Primary amines assessment of the test materials was necessary to ensure that a sufficient number of primary amines were revealed on the material. To visualize the distribution of primary amines, samples were washed 3 times with acetone and incubated in 5 mg/ml fluorescamine in acetone for 40 min in the dark. After washing 3 times with acetone to remove free dye, bound fluorescence was visualized by confocal laser scanning microscopy (λ=390 nm).

Upon a determination that a sufficient number of primary amine are available on the bone allograft, the bone allograft was coupled with 2 mg/ml Fmoc-[2-(2-amino-ethoxy)ethoxy]-acetic acid (Fmoc-AEEA) in DMF/diisopropylethylamine (DIEA, 100:1) in the presence of O-(7azabenzo-triazole-1-yl)-1,3,3-tetramethyluronium hexafluorophosphate (HATU), 2 h on shaker, RT, followed by washing 10 times with DMF. The linker was deprotected by treatment with 20% piperidine in DMF, 30 min, washed 10 times with DMF and a second linker coupled and deprotected as described above. After the final Fmoc deprotection, succinylation was completed with succinic anhydride and 4-dimethylaminopyridine (DMAP) added to triethylamine and tetrahydrofuran (THF) under anhydrous conditions and left for 24 hours. Rinses with THF and dH2O were carried out 3 times each. After the addition of 5 ml of dH2O and 1-ethyl-3-(3-dimethylaminopropyl) carbodiimide hydrochloride (EDC), n-hydroxy succinimide (NHS), and the mixture was stirred for 30 min. Triethylamine, was added in a dropwise fashion, along with a fourfold molar excess of doxycycline and was stirred for 24 hours. The graft was then extensively washed with DMF and incubated in ethanol and dH2O. Before antimicrobial challenge, grafts were incubated in PBS for at least 2 weeks to allow adsorbed reactants and antibiotic to elude.

Immunohistochemical detection of doxycycline. Control and doxycycline-modified-allograft (DOX-allograft) was washed 3× with Phosphate-Buffered Saline (PBS), blocked with 10% Fetal Bovine Serum (FBS) in PBS (blocking buffer), 1 h, and incubated with sheep anti-tetracycline (anti-TET) IgG (1:500, US Biologicals) in blocking buffer, room temperature (RT), 1 h. To remove unbound primary antibody, samples were washed 3× with PBS then placed on a shaker for 15 mins. This step was repeated one more time. The bone samples were then incubated with an AlexaFluor-488 donkey anti-sheep IgG secondary antibody (1:1000, Molecular Probes), RT, 1 h, and washed 3× with PBS followed by shaking for 15 mins. The samples were visualized by confocal laser microscopy.

Bacterial Culture and Challenge. Ten (10) mg dry weight of control and DOX-allograft samples were sterilized with 70% ethanol for 15 min, followed by washing 3× with PBS and 3× with trypticase soy broth (TSB). E. coli or S. aureus (Xen36 derived from ATCC®49525™; Caliper Life Science) were cultured in TSB, 250 rpm, 37° C., overnight. Using a 0.5 McFarland standard (A₆₀₀=0.10 yields ˜1×10⁸ cfu/ml), 1×10⁴ cfu/ml bacteria were incubated with the sterilized samples in TSB, 37° C. under static conditions for 6 hours.

Confocal Microscopy determination and review of the challenged materials. Upon harvesting, samples were washed 3× with PBS to remove non-adherent bacteria and stained with the Live/Dead BacLight™ Kit (Invitrogen; 20 min, RT). Fluorescent samples were visualized using optical sectioning in the Z-plane, followed by reconstruction using a confocal laser scanning microscope (Olympus Fluoview 300).

Scanning Electron Microscopy was further utilized to review and image the test bone materials. Samples that were prepared for SEM, were washed with dH2O, fixed with 4% paraformaldehyde, 1 h, and dehydrated with increasing ethanol dilutions (10-30-50-70-100% ethanol for 10 mins each) and vacuum dried overnight after incubation in Freon 113. Samples were then sputter-coated with gold and surfaces visualized using a Hitachi TM-1000 Tabletop Microscope.

Ultimately, after challenging the test materials efficacy of the DOX was assessed by determining whether the DOX bound to the bone samples resulted in a reduction of bacterial counts as compared to a control. Accordingly, after bacterial challenge was completed, the bone samples were gently washed 6× in PBS and sonicated 5 mins in 0.3% Tween-80 to suspend the adherent bacteria. After serial dilution, S. aureus and E. coli were plated on 3M® Petrifilms. Films were scanned and the colonies were counted using a macro in Adobe Photoshop CS3.

Cellular toxicity assays were also performed to test whether the cells being treated maintained normal cytoskeletal morphology. Accordingly, bone squares (1×1 cm) were sterilized with 70% ethanol, rinsed 3× with PBS, 3× with DMEM/F-12 containing 10% FBS (complete medium) and exposed to UV radiation for 20 min. 1 ml of 50,000 cells/mL human fetal osteoblasts (hFOBs) in complete medium were incubated on the sterilized samples for 48 hrs. Cells were then fixed in 4% paraformaldehyde, 10 min, RT, permeabilized with 0.1% Triton X-100 in PBS, 5 min, RT, and after incubation with blocking buffer, 30 min, stained with Alexafluor488-phalloidin (1:1000, Invitrogen) to visualize the cytoskeletal morphology and propidium iodide (1:100, Invitrogen) to visualize the nucleus, 30 min, RT. Stained cells were visualized using confocal laser scanning microscopy.

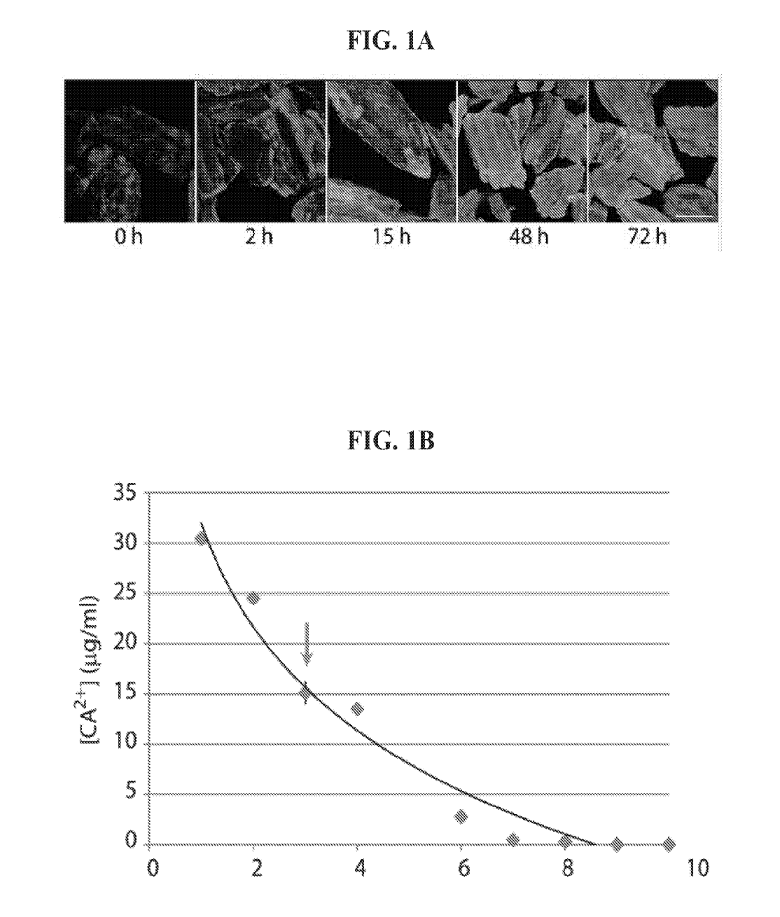

In view of the ability to attach DOX, evaluation of the underlying base material was necessary to identify materials that could be utilized as donor materials and be coupled to antibiotics using these strategies. Amines used for connecting to a linker and ultimately to an antibiotic are increased with demineralization of bone. The morselized bone was evaluated for the presence of primary amines that could be used as anchors for attachment of antibiotics. For example, as depicted in FIG. 1A, surface demineralization of morselized bone is depicted. Primary amines were further exposed by demineralization for 0-72 h, labeled with fluorescamine, and observed by confocal microscopy. Note that the fluorescent signal becomes more intense as a function of time, implying increased primary amine availability. Scale bar=400 μm. FIG. 1B depicts calcium content during demineralization. [Ca²⁺] in the EDTA bathing solution was determined by atomic adsorption spectroscopy. The graph shows the calculated value of [Ca²⁺] as a function of time of demineralization. Note that even after 3 days of demineralization (arrow), abundant Ca²⁺ still remains in the bone core. EDTA, ethylenediaminetetraacetic acid.

Fully mineralized bone was stained with fluorescamine, which binds to primary amines. Staining appeared patchy with areas of intense staining adjacent to areas showing little to no fluorescence, indicating that limited surface amines were available for chemical coupling (0 h, FIG. 1A). With as little as 2 h incubation in 12.5% EDTA, fluorescamine staining increased. By 15 h incubation in EDTA, fluorescamine staining was largely punctate and interspersed with large areas of intense staining. These intensely fluorescent areas increased in size, until by 72 h, fluorescence was uniformly intense over the surface of the morsels.

To determine the extent of demineralization during these treatments, Ca²⁺ content of the EDTA solution over the demineralization course of 10 days was monitored by atomic adsorption spectroscopy (FIG. 1B). The Ca²⁺ concentration of the EDTA solution was highest after the first day of demineralization, reaching 30˜μg Ca²⁺/mL. Ca²⁺ concentrations then decreased gradually until they were no longer detectable by day 10.

Importantly, the Ca²⁺ content extracted from the bone during the first 3 days of incubation (arrow) is ˜60% of the total Ca²⁺ content of the bone. The effect of demineralization on amine availability was directly measured using the Ninhydrin assay, which forms a colored adduct in the presence of primary amines.¹⁸ The untreated, mineralized bone contained ˜1×10⁻⁵ mol of primary amine/mg of morselized bone; demineralization for 3 days increased the amount of available amines ˜4-fold (˜3.8×10⁻⁵ mol/mg bone).

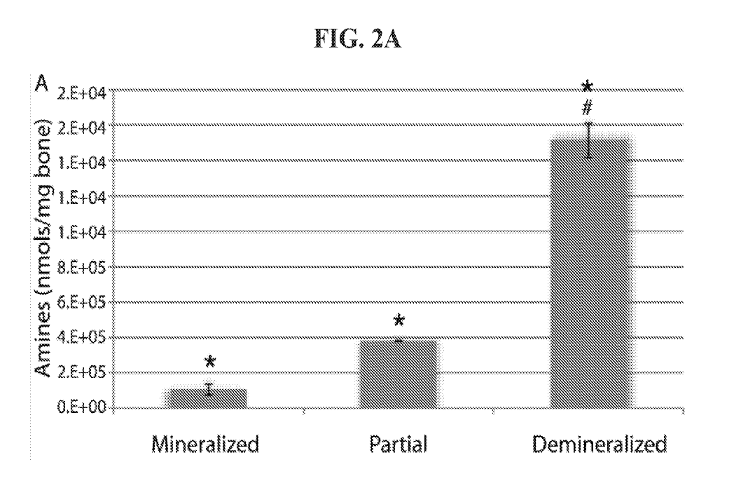

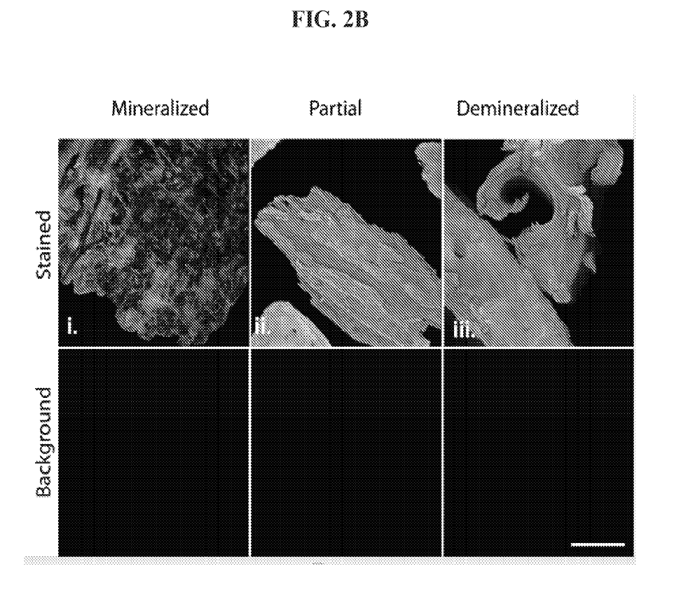

FIG. 2 depicts amine content and coverage of bone. In FIG. 2A, the primary amine content of bone after 0, 3, or 10 days of demineralization, was determined using the Ninhydrin assay. Fully mineralized bone yielded the lowest concentration of amines. Bone that was treated with 12.5% EDTA for 3 days (partial) had significantly increased levels of primary amine availability but was still significantly less than that of fully demineralized bone (*ρ<0.005 from mineralized, #ρ<0.005 from partial). In FIG. 2B, primary amines on the bone surface were observed by fluorescamine staining. Shown are (i) fully mineralized bone, (ii) partially demineralized bone, and (iii) fully demineralized bone. Scale bar=400 μm.

Indeed, as described above and as depicted in FIGS. 2A and 2B, demineralized bone had the greatest number of available primary amines, at ˜1.8×10⁻⁴ mol/mg bone (FIG. 2A). Indeed, it is necessary to have more amines than antibiotics to ensure a sufficient number of antibiotics bound to the material. In preferred embodiments, a ratio of 6:1 primary amines to the number of antibiotics necessary to be attached. When parallel samples were stained with fluorescamine (FIG. 2B), mineralized bone had the patchy staining pattern noted in FIG. 1. Bone demineralized for 3 days had a fluorescamine intensity similar to that of fully demineralized bone (FIG. 2B, ii vs. iii). On the basis of our data from FIG. 1, this staining pattern indicated that partial demineralization predominantly released the readily available mineral, that is, surface mineral. Thus, this 3-day treatment (referred to as “partially demineralized bone”) was used as a starting point for all further modifications.

Allografts are used in applications other than orthopaedic. For instance, saphenous veins are used in vascular surgery, xenograft heart valves are used in cardiology applications, and allograft tendons may be used. All of these allograft materials are subject to infection like allograft bone. To extend the application, we have use saphenous vein as a substrate and successfully used the chemistry described herein to bond VAN to the saphenous vein. This vein resisted colonization by S. aureus proving that chemistry described in this application can be applied to many allograft/xenograft tissues.

However, and by example of a bone allograft as the antibiotic carrier tissue, using partially demineralized bone (which is used as control bone in all subsequent experiments), two 2-(2-aminoethoxy)-ethoxy]-acetic acid linkers were sequentially coupled to the allograft, followed by attachment of DOX via its carboxylic acid group to the linkered surface (Scheme, FIG. 3). Morselized allogenic bone was cleaned by sonication in dH₂O and partially demineralized with 12.5% EDTA for 3 days. After washing twice in water with sonication, it was sonicated for 30 min twice in DMF. It then underwent two rounds of coupling with Fmoc-AEEA in DMF/DIEA, washed 10 times in DMF, and deprotected by treatment with 20% piperidine in DMF for 30 min, followed by an additional 10 washings in DMF. This procedure was followed for every addition of an Fmoc-AEEA linker so that a chain of 1-4 linkers was added to the allograft surface. After the final Fmoc deprotection, samples were either coupled with 10 mg/mL clinical-grade VAN in DMF/DIEA in the presence of HATU or washed extensively with DMF and dH₂O, or they were coupled with doxycycline.

Specifically, the deprotected allograft-AEEA surface was reacted with succinic anhydride in DMAP+TIEA for 24 hours, washed in THF three times, followed by washing with water three times. These were then reacted with EDC+NHS. VAN, vancomycin; DMF, dimethylformamide; Fmoc-AEEA, Fmoc-[2-(2-amino-ethoxy)-ethoxy]-acetic acid; DIEA, diisopropylethylamine; DMAP, 4-Dimethylaminopyridine; TIEA, triethylamine; EDC, I-ethyl-3-(3-dimethylaminopropyl) carbodiimide hydrochloride: N-hydroxy succinimide in water, with stirring for 30 min, followed by a dropwise addition of TIEA. Doxycycline was then added, and incubated with robust stirring for 24 hrs. The reaction products were washed with DMF>10 times, incubated in ethanol overnight with stirring, and incubated with water overnight with stirring.

During the synthetic process, allograft is exposed to high concentrations of the antibiotic to be coupled, allowing VAN adsorption as well as covalent bonding. Therefore, we monitored VAN elution from VAN-allograft by placing sample morsels on a uniform S. aureus lawn daily and comparing the resulting zones of inhibition with those from a standard 10 μg/mL VAN adsorbed to control bone. Even longer incubation times have resulted with doxycycline and tetracycline, where 2 weeks may be required to elute the adsorbed antibiotic. This is actually a positive associated with this class of antibiotic as they are calcium-binding antibiotics and thus have prolonged stability in the calcium-rich bone matrix, which would give both an elution and permanent protection over the course of the first month.

Through the process of demineralizing the bone, primary amines are generally present in substantially uniform concentrations (with local irregularities) on the surface of the bone. For example, in both the VAN and DOX tests, the antibiotics, after being coupled are present all over the bone surface, in a substantially uniform coverage to ensure that there are not pockets or areas on the surface that do not possess sufficient number of antibiotics to prevent or reduce infection.

Furthermore, tests show that the bonds between the attached antibiotics and the linker, and the linker and the primary amine are strong. After the 3-5 days of elution, in an example using VAN, the VAN allograft was evaluated for VAN distribution using anti-VAN antibodies. By immunofluorescence, intense VAN staining was apparent on the VAN-allograft whereas control unmodified bone showed only a diffuse background fluorescence. Staining of the VAN allograft with secondary antibody alone showed no appreciable signal, confirming the specificity of the observed staining and thus confirming that the VAN was not eluted from the material.

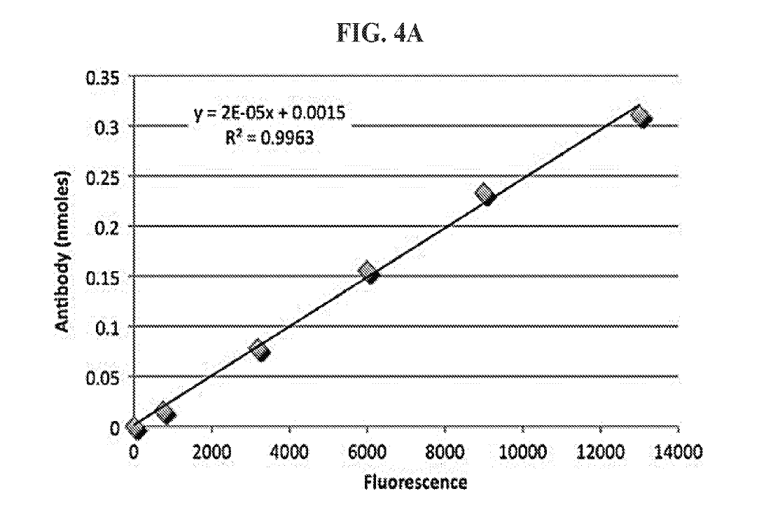

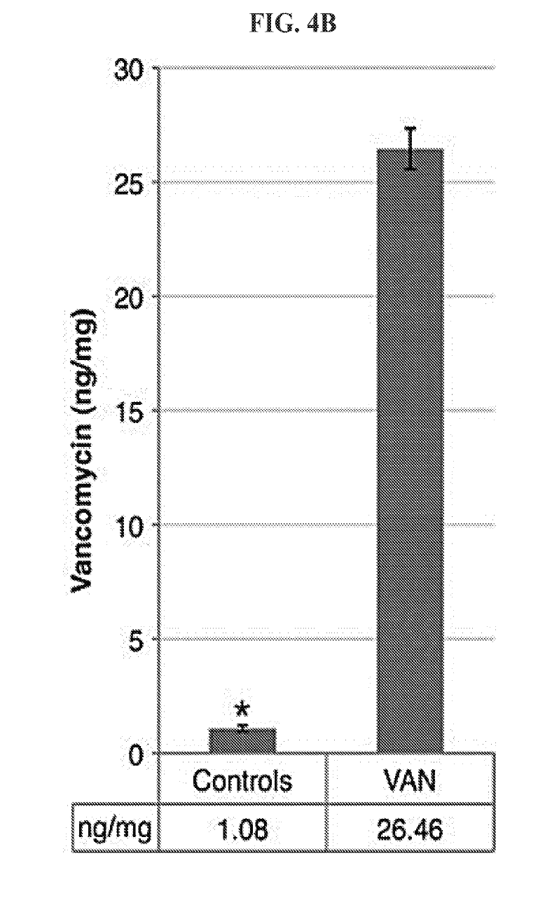

To approximate VAN amounts bonded to the bone matrix, anti-VAN antibodies were dissociated from both control and VAN-allograft by treatment with 7M urea. FIG. 4 depicts measurement of immobilized VAN on VAN-allograft. FIG. 4A depicts a standard curve for fluorescence of AlexaFluor488 secondary antibody (0-0.2 μg/mL) in the 7M urea extraction buffer. Note that the R² value is close to 1. FIG. 4B depicts a graphical representation of amounts of antibody recovered from control or VAN-allograft. Approximately 26 ng of immobilized antibiotic per mg of morselized bone was calculated from the standard curve. Controls showed a low VAN level due to limitations of the assay. *ρ<0.005. Similar concentrations were also found with DOX. Alternatively, the concentration can be defined as corresponding to a concentration of 1.53 molar antibiotic on the bone.

As described above, a secondary antibody standard curve (FIG. 4A) was generated; after conversion of released amounts, control bone released antibody equivalent to 1.08 ng VAN/mg bone (background), whereas about 26 ng VAN/mg bone was calculated for VAN-allograft (FIG. 4B).

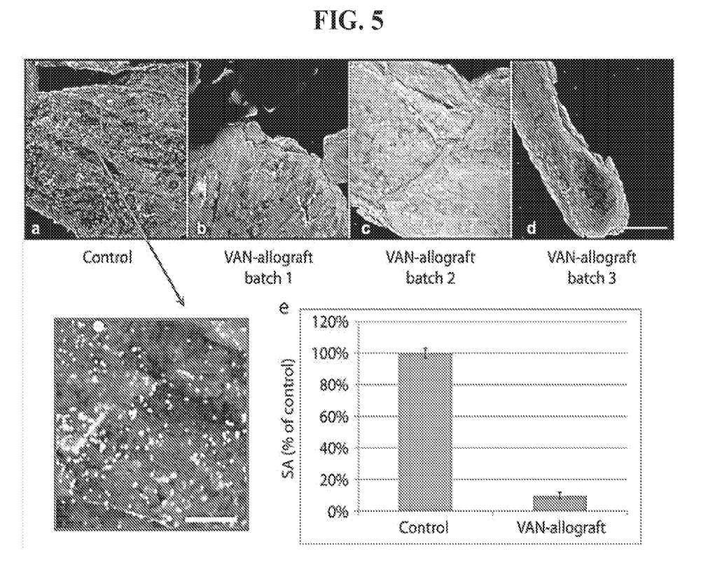

Testing of the bound antibiotics was fruitful in defining that VAN-allograft resists colonization by S. aureus. FIG. 5 depicts that VAN-allograft resists S. aureus colonization. Control or VAN-allograft were challenged with S. aureus (Cᵢ=10⁴ cfu) for 6 h and adherent bacteria were observed using the Live/Dead assay. FIG. 5a depicts abundant viable and some dead bacteria detected on control samples that uniformly covered the bone surface. (a, insert) Bacteria are also seen organizing in clusters producing areas of Live/Dead (Syto9 and Propidium Iodide) stain uptake that may mark the initiation of biofilm formation. (b-d) In sharp contrast, few, if any, dispersed bacteria were present on VAN-allograft from three different synthetic batches. VAN-allograft consistently appeared devoid of bacterial colonization and only exhibited a uniform yellow appearance produced from the natural fluorescence of the bone surface itself. (e) Adherent bacteria were released from the surface of bone by sonication and quantified by serial dilution and plating. VAN-allograft was able to resist colonization and significantly reduced the bacterial load on bone grafts by >90%. *ρ<0.05. Scale bars: (a-d) 200 μm (a, insert) 50 μm.

Accordingly, the ability of VAN-allograft to resist colonization with S. aureus was confirmed. After a 6 h S. aureus challenge (Cᵢ=10⁴ cfu), control bone was abundantly colonized, with the surface of the morsels occupied by the fluorescently stained S. aureus (FIG. 5a). Bacteria were organized in microcolonies (individual bright green dots) and in continuous clusters that are indicative of biofilm formation (FIG. 5, inset). VAN-allograft from three separate syntheses was able to resist colonization and appeared free of bacterial attachment (FIG. 5b-d). In addition, adherent bacteria were recovered and counted; bacterial loads were reduced by up to 90% on VAN-allograft as compared to controls (FIG. 5e).

Amine concentrations determine the number of antibiotic molecules/mm² of the surface. Whereas partially demineralized bone has sufficient amines to result in a high enough density of VAN to become antibacterial, allograft bone with fewer amines, or artificial extracellular matrix with fewer amines do not achieve an antibacterial surface. Thus, in certain embodiments, the materials may be defined by a threshold of amine concentrations, such as about 3.8×10⁻⁵ moles/mg bone, is required to achieve an antimicrobial surface. Accordingly, concentrations of amines less than about 3.8×10⁻⁵ moles/mg bone results in an insufficient number of amines to bind the antibiotics to the tissue.

In certain preferred embodiments, the concentration of the amine groups on the surface of the bone graft is important to ensure sufficient concentrations of the antibiotic or other bound molecule or pharmaceutical material. Accordingly, the process to decellularize the bone is important in certain embodiments to control the formation of free amine groups on the surface of the bone.

In certain situations, low formation of amine groups results in binding of lower numbers of pharmaceutical molecules. However, in other situations, an overabundance of amine groups may result in inefficient binding to the bone surface. In such a situation, the linkers may far exceed the amount of antibiotic, with the terminal amine of the linkers influencing the antibacterial properties of the surface

In other embodiments, it may be appropriate to perform a first application on a first antibiotic material, wherein the bone is only partially demineralized and then bound with an antibiotic of interest. The antibiotics can then be chemically protected and a second demineralization process may proceed to reveal additional amine groups, and a second antibiotic or other material of interest can then be bound to the bone or other tissue.

In other embodiments, it may be possible to simply demineralize the bone to reveal the amine groups. Subsequently linkers are bond to the surface and then a mixture of pharmaceutical molecules or materials are bound to these linkers simultaneously. This provides that more than one antibiotic or other material can be bound to the linker at this stage. Rates of binding and relative concentrations of the materials can be modified by the size of the materials relative to one another as well as the initial concentrations when binding to the bone.

The microbiological and immunohistochemical studies indicate that the coupling chemistry maintained most or all of the spectrum of VAN activity. Chemical modification of this construct with VAN was performed on multiple independent batches of morselized bone to ensure reproducibility not only in successful attachment of the antibiotic but also in its antibacterial activity. Before evaluation of the VAN-allograft, it was incubated in aqueous medium until no zones of inhibition (indicating antibiotic elution) were produced on uniform bacterial lawns.

Immobilization instead of impregnation of antibiotic onto the grafts has several implications. First, the antibiotic does not dissociate from the graft and therefore does not get depleted. Second, it is expected to stay on the surface of the bone, providing long-term protection that may withstand multiple bacterial challenges. Finally, no systemic toxicity is expected since the antibiotic is limited to the surface of the graft where it inhibits the attachment of bacteria. Even if you eluted all of the antibiotic on the graft at once, the actual concentration on the graft is sufficiently low so that it would be 100-1000× less than the amount needed to inhibit bacterial growth and below concentrations that would be expected to foster antibiotic resistance. Toxicity usually starts occurring at 100-1000× more than the minimal inhibitory concentration. Thus the materials described herein provide highly efficacious mechanisms to prevent infection on bone or tissue material use in an implant.

Even under conditions where the bone matrix is resorbed, antibiotic release would be small and therefore not cause toxicity. The predicted long-term protection of the chemically bonded VAN-allograft was borne out in studies examining the ability of the VAN-allograft to withstand a bacterial challenge. VAN-allograft resisted bacterial colonization and, when compared to untreated controls, reproducibly reduced the bacterial load by >90%. The Staphylococci that were used for this study represent the main culprit in graft-associated infections and account for 36% to 38% of all allograft infections.^{11,12} Further, these bacteria are notoriously efficient biofilm producers making eradication of established infections nearly impossible.²⁷ By inhibiting the initial step of attachment, the biofilm formation can be prevented in whole or in part.

Whereas most deep infections associated with normal operative procedures are Gram-positive bacteria that are sensitive to VAN, there are times when Gram-negative bacteria, such as Escherichia coli (E. coli), play an important role in implant associated infection. VAN is a peptidoglycan antibiotic that inhibits cross-linking of the Gram-positive bacterial cell wall; Gram-negative bacteria do not have this cell wall and VAN cannot inhibit their growth. Thus, the VAN-allograft surfaces do not protect against Gram-negative bacteria. Thus, we looked at the tetracycline family of antibiotics. Tetracyclines, such as tetracycline and doxycycline (as well as the related glycylcycline family which includes minocycline and tigecycline) are broad spectrum antibiotics that inhibit protein synthesis by reversible binding to the ribosome. Furthermore, TET and DOX can perturb surface bacterial activity, which may provide a beneficial mechanism in the embodiments described herein. Attachment of VAN to a linker occurs using HATU chemistry and couples its carboxylic acid to the tethered amine on the surface. In contrast, tethering of DOX relies on an amide coupling at an amine mediated through succinylation of the growing surface-bound linkers.

As both VAN and DOX, as well as other classes of antibiotics disclosed herein are superior to others for certain classes of bacteria, it may be suitable in certain embodiment to utilize a bone graft comprising a combination of bound antibiotics, wherein the combination of antibiotics provides for effective treatment and protection against a variety of bacteria. Further embodiments are advantageously directed to embodiments using a combination of antibiotics and bone formation products that are each bound to the surface of the bone, whereby antibacterial properties prevent infection and bone formulation compounds promote healing and restoration of the bone.

Indeed, as depicted and described in the embodiments herein, colonizing bacteria are reduced or limited when antibiotics are attached to the modified tissues. The clear reduction in bacterial load is sufficient to cause a real difference in the outcome of infection. Therefore, a method provides for a process that demineralizes bone to reveal primary amines, add functional linker groups to the primary amines, and then attach one or more antibiotics, or other suitable pharmaceutical material to the linker groups. The linker groups provide sufficient space between the active pharmaceutical material (i.e. antibiotic) and the tissue material to ensure that the pharmaceutical material works away from the surface thus ensuring activity of the pharmaceutical material is not compromised by blocking active sites. Furthermore, the linker groups ensure that the biologic materials are attacked prior to attaching to the surface of the bone, where it becomes more difficult to remove or prevent infection.

The materials, and methods of making such materials, are further applicable to other decellularized grafts, such as vascular grafts. Since the tethering relies on primary amine availability in the collagenous extracellular matrix, the same technology can be extended to prosthetic heart valves,^{28, 29} decellularized hearts,³⁰ and other native nonviable tissues.

Interestingly enough, certain materials have proven to be incompatible with the methods described herein, based upon the insufficient number of primary amines in the molecule. For example, in attempts at tethering antibiotics to pure fibronectin, it was discovered that there was an insufficient numbers of amines in the molecule. Indeed, it is suspected that with collagen, because it exists as a triple helix, which assembles into fibrils, the surface amines may be different than those counted up in the linear collagen molecule. Furthermore, it is decorated by other ECM proteins. Accordingly, tethering directly to fibronectin materials, or those having insufficient amine concentrations mean that such materials would need likely need a different attachment chemistry to reach the same concentration of antibiotics as are bound in the examples described herein.

Importantly, the methods described in this report, therefore, can be used to tether other antibiotics, chemotherapeutics, and chemotactic, osteogenic, angiogenic, and antithrombotic factors to the graft and other suitable materials having a sufficient number of primary amines on the surface of the material. Thus, covalent modification of native tissues represents an important advance in enhancing functionality and preventing the devastating complications of uncontrolled infections.

The linking process relies upon the presence of sufficient numbers of primary amines, which could serve as anchors for chemical synthesis, increased with limited demineralization in bone tissues; other tissues such as venous grafts have sufficient exposed primary amines to allow successful tethering of antibiotics. Preparation of materials to ensure sufficient numbers of these primary amines is necessary to ensure efficacy of the materials. Using these amines, up to 4 linkers and VAN, TET or DOXY are linked to bone using Fmoc chemistry. It is worth noting that these antibiotics act by reversibly binding to their targets. Therefore, the surface is able to “regenerate” after release of the target. Other antibiotics that have been successfully linked include kanamycin, ceftriaxone, and gentamicin. However, these antibiotics (aminoglycosides, cephalosporins) act and bind through covalent modification of their targets so the surface did not regenerate after interaction with the bacterium.

Therefore, as described in the embodiments herein, through immunohistochemistry, antibiotic can be abundantly tethered to the surface of the allograft; based on elution and measurement of bound antibody, this coupling, in certain embodiments, yielded at least ˜26 ng VAN/mg bone. The coupled VAN as well as the coupled TET or DOXY appeared to be permanently bound to the allograft, as it showed no elution in a disk diffusion assay, and, importantly, resisted colonization by Staphylococcus aureus challenges. This chimeric construct represents a new generation of antibiotic-modified allografts that provide antibacterial properties.

In certain embodiments, the products made by the processes defined herein are suitable for use in certain medical settings, wherein the use of the products is sufficient to prevent or reduce the incidence or occurrence of bacterial infection.

### Examples

The following examples are provided as non-limiting examples of the various processes and products described herein.

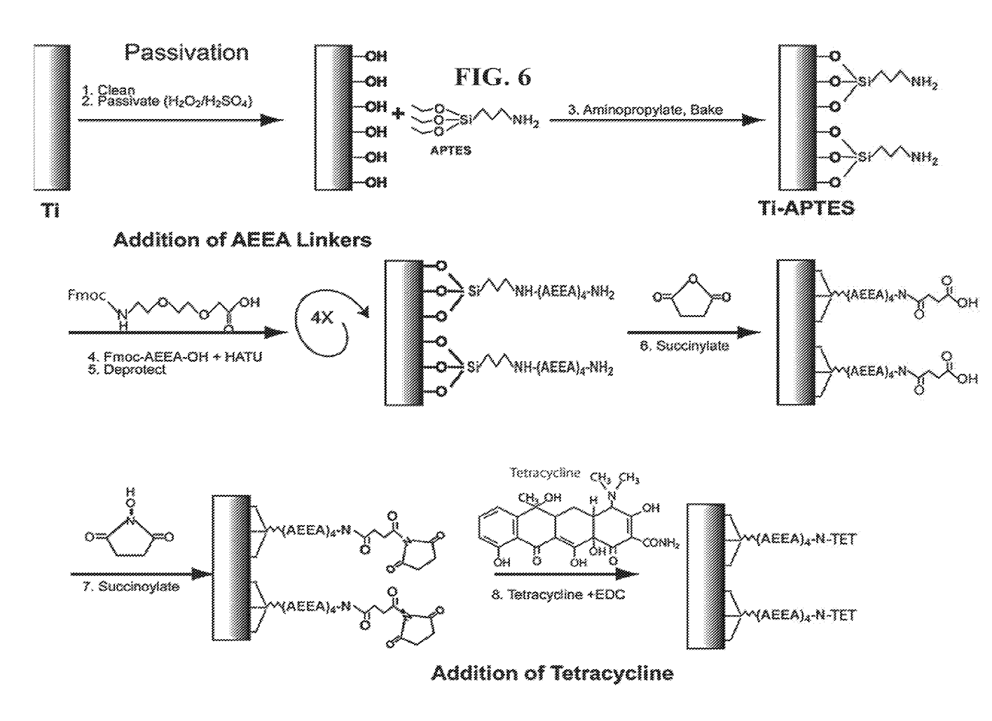

Bone is comprised of a complex combination of extracellular matrix proteins and mineral. Each of the extracellular matrix proteins display primary amines on their surfaces that could, in theory, be used as an anchor point for the bonding of Doxycycline. Surface availability of primary amines, however, seems to be directly related to the degree of mineralization⁵. Thus, as previously described, as a first step, we partially demineralized bone with EDTA for 3 days. This mild treatment exposes the underlying matrix that is mainly comprised of collagen and that bears the amines that we target (FIG. 6—synthetic scheme).

This demineralized bone was coupled to two AEEA linkers; these first several steps presents an amine rich surface that can be used for coupling of carboxylic acid-containing drugs via HATU chemistry as described for VAN, or using succinylation followed by reaction with carbodiimide, to covalently bond Tetracycline (FIG. 6—synthetic scheme) or Doxycycline. To assess antibiotic attachment and distribution, modified, morselized allograft was incubated with anti-tetracycline antibody, followed by a fluorescent secondary antibody; this antibody was chosen as its epitope does not include the amide group assumed to be used for coupling of the Doxycycline to the linkers. After staining with anti-Tet antibodies, fluorescence was apparent over the surface of the morsels, suggesting successful covalent attachment of DOX to bone. Control samples showed only weak, diffuse staining representing the natural fluorescence of bone.

To detect possible alterations of surface morphology resulting from chemical modification with Doxycycline, the appearance of DOX-bone was compared to that of control bone using scanning electron microscopy. Based on these micrographs, the surface of bone appeared to become slightly smoother after each of the different synthetic steps but overall topographical characteristics remained unchanged.

Antimicrobial Activity of the Constructs

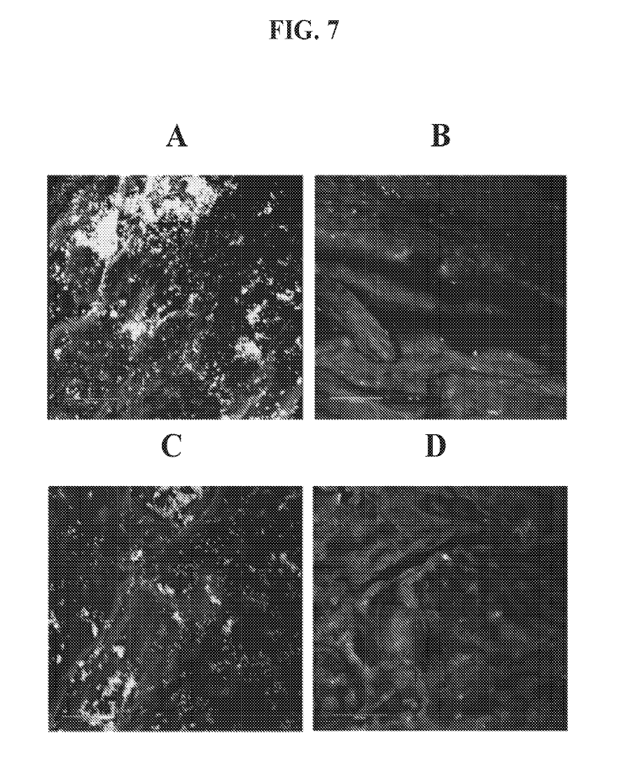

To determine whether the covalently bonded Doxycycline inhibited bacterial colonization, DOX-bone and control bone allograft were challenged for 6 hours with E. coli and S. aureus (Ci=10⁴ CFU) under static conditions. The bone samples were then treated with Live/Dead stain and viewed under confocal laser microscopy. Control bone challenged with S. aureus exhibited multiple green foci, representing bacterial colony formation, as well as patchy areas of increased fluorescent signal typical of biofilm formation (FIG. 7a). Control bone that was challenged with E. coli demonstrated a similar pattern of colonization as that of S. aureus, with slightly smaller multiple green foci and areas of concentrated fluorescence indicating colony formation and biofilm (FIG. 7c). DOX-bone challenged with S. aureus (FIG. 7b) and E. coli (FIG. 7d), appeared free of bacterial colonies and only showed background fluorescence.

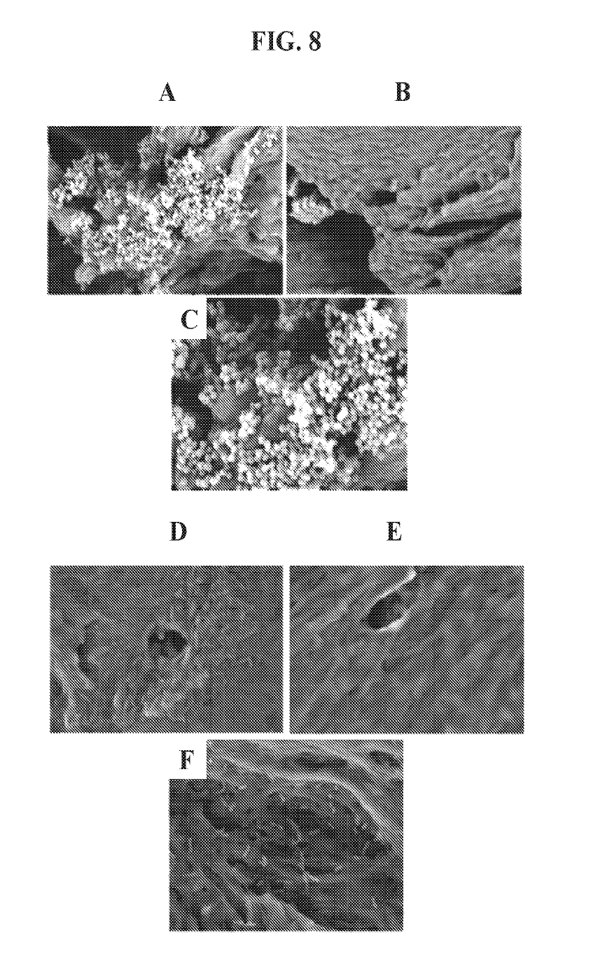

Control surfaces were then examined by SEM, which revealed that natural topographical niches, such as Haversian and Volckmann's canals, were consistently filled with S. aureus and E. coli colonies. When examining the S. aureus challenged control surfaces at 1500× magnification, clusters of spherical structures could be seen attached to the surface of the bone morsels (FIG. 8a). At higher magnification (5000×), they were recognizable as bacteria (˜1 m in diameter) that aggregated in grape-like clusters characteristic of S. aureus, as well as exhibiting areas of overlying slime matrix characteristic of biofilm (FIG. 8b). Similarly, surfaces that were challenged with E. coli demonstrated uniform rod-like structures that appeared to be organized in less dense colonies than S. aureus and can even sometimes be seen as individual bacteria (FIG. 8d). When examined at a lower magnification, some of the clustered bacteria are covered by a glycocalyx matrix (FIG. 8e). On the other hand, the surface of the DOX-allograft that was challenged with either S. aureus (FIG. 8c) or E. coli (FIG. 8f) appeared free of bacterial colonies.

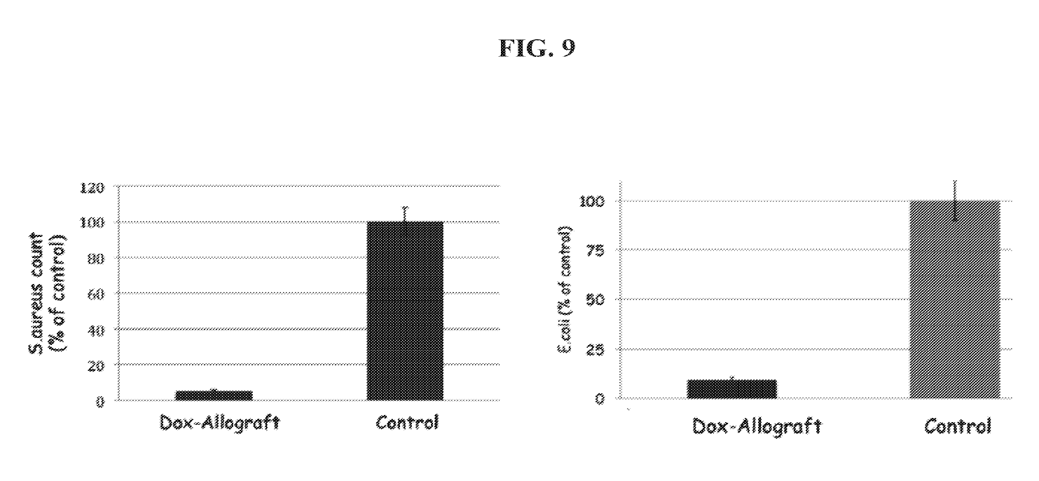

The efficacy of covalently bonded Doxycycline was confirmed by colony counts. After sonication and plating, recovered bacterial colonies were counted for S. aureus and E. coli on DOX-allograft and controls. DOX-allograft was able to resist colonization for both bacterial strains and significantly reduced the bacterial load on bone grafts by ˜95% for S. aureus (FIG. 9a) and ˜90% for E. coli (FIG. 9b).

Cellular Biocompatibility Assay

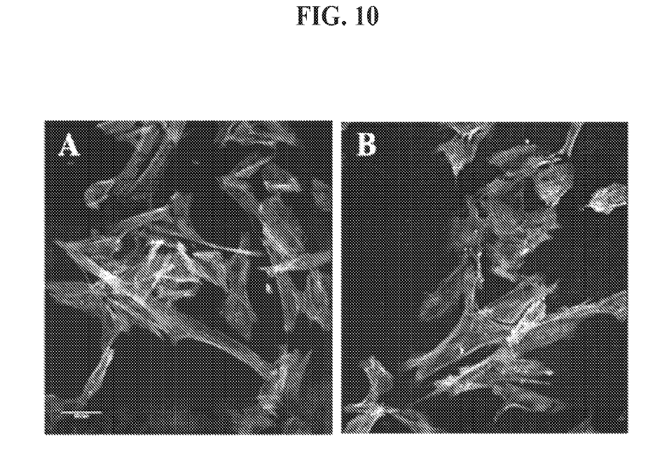

Biocompatibility of the DOX-allograft was assessed using human fetal osteoblast-like cells (hFOBs)¹¹. After 48 hrs in culture, hFOBs on the control bone appeared to have either an elongated or a trapezoidal shape, both of which are characteristic of well-adhered osteoblastic cells (FIG. 10a). On the DOX-allograft, hFOBs appeared similar in size and shape to that described for control bone (FIG. 10b). Overall actin cytoskeletal architecture appeared similar to that evident on the control samples. It is worth noting that normal nuclear size and number, as detected by propidium iodide staining, were present in both fields.

Similarly, the DOX bound grafts were evaluated for the bacteriostatic properties of the Doxycycline-bonded surface. The ability of the Doxycycline-allograft to resist colonization by E. coli was characterized by challenging the surface with 10⁴ CFU E. coli; the Doxycycline-modified allograft consistently decreased bacterial colonization by ˜90%. This high initial inoculate of 10⁴ CFU provides a measurable colonization on Doxycycline allograft, even though the number of organisms used for these studies are greatly in excess of what would be expected clinically.

To test how the DOX-allograft performed when compared to more conventional approaches, Doxycycline adsorbed onto allograft was compared with an engineered Doxycycline-allograft in which Doxycycline had been covalently bonded to the bone matrix as described herein. Initially, the two systems were examined for duration of antibiotic elution and for protection against colonization by E. coli.

Whereas samples with the adsorbed Doxycycline showed release of antibiotics up to day 15, the Doxycycline-allograft showed no elution. Furthermore, the Doxycycline-allograft, which retained its covalently bonded antibiotics, was able to provide protection for up to 4 months, a far longer time period than simple adsorption in which the antibiotic depletes over time.

A problem with elution of antibiotics, as described above, is the creation of concentration gradients and the development of resistance. At the end of elution, exposure of bacteria to sub (but near)-inhibitory concentrations of antibiotics has been suggested to increase the risk of resistance strains. In contrast, DOX-allograft showed no significant antibiotic elution suggesting that, with this system, bacteria are not expected to be exposed to sub-inhibitory concentrations of antibiotic, nor develop resistance. Importantly, antibiotic immobilization suggests that no antibiotic is expected to be found systemically, overcoming toxicity concerns often raised with elution systems: drug interactions, adverse effects, disruption of commensal flora.

Finally, these modified bone allografts need to be biocompatible. The tethered Doxycycline neither alters the actin cytoskeletal architecture nor the morphological characteristics of osteoblast-like cells. It is very important that any modification that renders the graft bactericidal not alter the underlying properties of the bone. The detrimental effects of high concentrations of solution antibiotics on mammalian cells are well-established in the literature, but little is known about the effects of immobilized antibiotics. Using the methods and materials described herein, the osteoblast-like cells that were cultured on Doxycycline-allograft and visualized by actin cytoskeletal staining did not show any morphological differences to those cultured on control bone. In fact, low Doxycycline concentrations (1-5 g/ml) have been shown to stimulate the proliferation of osteoblast-induced bone marrow cells and enhance maturation and differentiation of osteoblastic cells. The mechanism by which Doxycycline exerts its favorable effect on osteoblasts is largely unknown but is most likely due to indirect mechanisms. Specifically, chelation of calcium with Doxycycline, thereby diminishing free calcium concentration, might inhibit MMP activity since these enzymes are cation-dependent. Therefore, these mechanisms may remain active with immobilized Doxycycline, and the DOX-allograft could favor osteoblast proliferation on its surface. In that situation, such constructs can be doubly advantageous, not only reducing infection but also favoring bone formation. Such an advantage provides for significant advances in bone restorative procedures that not only fight off infection but that promote the healing and bone formation process in vivo.

Substantial effort has also been made in attaching Vancomycin (VAN) to bone or other tissue. Allograft preparation Cancellous/cortical human bone granules 0.5-2 mm (Musculoskeletal Transplant Foundation) were washed and sonicated with dH₂O until washings were clear. Indeed, the bone should be hydrated before proceeding, as lyophilized bone does not demineralize appropriately to reveal amine groups. Where indicated, samples were partially demineralized with 12.5% EDTA (pH 7) at room temperature (RT) for 3 days with shaking, and daily replacement of the EDTA. Before synthesis, all samples were washed with dH₂O and sonicated twice for 30 min in dimethylformamide.

Assessment of Primary Amines Morselized bone was incubated with 12.5% EDTA (pH 7) at RT for 10 days with shaking; eluent containing dissolved calcium was replaced daily with fresh EDTA. After its dilution with 0.5N HCl and 0.1% LaCl₃, this eluent was assessed by atomic adsorption spectroscopy to determine the Ca²⁺ content. To determine surface amine distribution, these morselized bone samples were washed thrice with acetone and incubated in 1 mg/mL of fluorescamine in acetone for 40 min in the dark. After washing thrice with acetone to remove free dye, bound fluorescence was observed by confocal laser scanning microscopy. To measure total amounts of primary amines, 10 mg dry weight of morselized allograft was boiled in a water bath for 15 min in 2 mL of solution comprised of 100 μL 3.5 M SnCl₂, 100 μL 1M Na₃C₆H₅O₇.2H₂O pH 5, 1 ml 4% Ninhydrin in ethanol and 800 μL dH₂O. The absorbance of the supernatant was measured and the molar concentration of primary amines calculated (ε₅₇₀=1.5×10⁴ [M-cm]⁻¹).

Allograft Modification.

Washed allograft was coupled with 10 mg/mL Fmoc-[2-2amino-ethoxy)-ethoxyl]-acetic acid in DMF/diisopropylethylamine (100:1) in the presence of O-(7-azabenzo-triazole-1-yl)-1,1,3,3,-tetramethyluronium hexa-fluorophosphate (HATU), 2 h, room temperature, followed by washing 10 times with DMF. The linker was deprotected by treatment with 20% piperidine in DMF, 30 min and washed 10 times with DMF, and a second linker was coupled and deprotected as above. After the final Fmoc deprotection, samples were coupled with 10 mg/mL clinical-grade VAN (American Pharmaceutical Partners, Schaumburg, Ill.) in DMF/diisopropylethylamine (100:1), in the presence of HATU, for 12-16 h. The modified bone was washed extensively with DMF and dH₂O.

Immunohistochemical Detection and Quantization of VAN Control or VAN-allograft was washed thrice with PBS, blocked with 10% fetal bovine serum in PBS (blocking buffer) for 1 h, and incubated with rabbit anti-VAN IgG (1:500; US Biologicals, Swampscott, Mass.) in blocking buffer at 4° C. for 12 h. Samples were washed thrice with PBS and incubated with an AlexaFluor 488-goat anti-rabbit IgG secondary antibody (1:1000; Molecular Probes/Invitrogen, Carlsbad, Calif.) at RT for 1 h, and washed thrice with PBS. These fluorescently stained samples were observed by epifluorescence, or to elute the bound antibody, 30 mg of these control and VAN-allograft samples was incubated with 1 ml of 7M urea with slow rocking at 4° C. overnight. The eluted fluorescent antibody was measured (λ_{Ex}=494 nm; λ_{Em}=520 nm, TECAN plate reader), and VAN concentrations were calculated by comparison to a standard curve of known concentrations of the secondary antibody in 7M urea.

Bacterial Culture and Challenge.

Ten milligrams dry weight of control or of VAN-allograft was sterilized with 70% ethanol for 15 min, and washed thrice with PBS and thrice with trypticase soy broth (TSB). S. aureus (Xen36 derived from ATCC 49525; Caliper Life Science, Hopkinton, Mass.) were cultured in TSB at 250 rpm and 37° C. for 12-14 h (overnight culture). Using a 0.5 McFarland standard (a turbidity measure in which A₆₀₀=0.10 for ˜1×10⁸ cfu/mL), 1×10⁴ cfu/mL bacteria were incubated with the sterilized samples in TSB at 37° C. under static conditions. Upon harvesting, samples were washed thrice with PBS to remove nonadherent bacteria, and either (1) stained with the Live/Dead BacLight™ Kit (based on the differential membrane permeability of Syto9 and propidium iodide; Invitrogen, Carlsbad, Calif.; 20 min, RT) and fluorescent bacteria were observed with confocal laser microscopy (Olympus Fluoview 300, Center Valley, Pa.), or (2) washed three more times and sonicated in 0.3% Tween-80 for 5 min to suspend adherent bacteria, which were then serially diluted, and suspended bacteria were plated on 3M® Petrifilms (3M Microbiology, St. Paul, Minn.). Petrifilms were scanned and colonies counted using a macro in Adobe Photoshop CS3.

It is to be understood that the scope of the embodiments is not to be limited to the specific examples described above. As will be apparent to one of ordinary skill in the art, the embodiments of the invention may be practice other than as particularly defined and still be within the scope of the accompanying claims.

### BIBLIOGRAPHY

1. Centers for Disease Control and Prevention. www.cdc.gov. 2010.
2. Enneking, W. F., Mindell, E. R., Burchardt, H., and Tomford, W. Allograft safety and ethical considerations: editorial comment. Clin Orthop Relat Res 435, 2, 2005.
3. Lewandrowski, K., Gresser, J., Wise, D., and Trantol, D. Bioresorbable bone graft substitutes of different osteoconductivities: a histologic evaluation of osteointegration of poly(propylene glycol-co-fumaric acid)-based cement implants in rats. Biomaterials 21, 757, 2000.
4. Boyce, T., Edwards, J., and Scarborough, N. Allograft bone. The influence of processing on safety and performance. Orthop Clin N Am 30, 571, 1999.
5. Souli, M., and Giamarellou, H. Effects of slime produced by clinical isolates of coagulase-negative staphylococci on activities of various antimicrobial agents. Antimicrob Agents Chemother 42, 939, 1998.
6. Buttaro, M., Comba, F., and Piccaluga, F. Vancomycin-supplemented cancellous bone allografts in hip revision surgery. Clin Orthop Relat Res 461, 74, 2007.
7. Keidar, Z., Engel, A., Hoffman, A., Israel, O., and Nitecki, S. Prosthetic vascular graft infection: The role of 18F-FDG PET/CT. J Nucl Med 48, 1230, 2007.
8. Chang, J., Calligaro, K., Ryan, S., Runyan, D., Dougherty, M., and Stern, J. Risk factors associated with infection of lower extremity revascularization: analysis of 365 procedures performed at a teaching hospital. Ann Vasc Surg 17, 91, 2003.
9. Costerton, J. W., Stewart, P. S., and Greenberg, E. P. Bacterial biofilms: a common cause of persistent infections. Science 284, 1318, 1999.
10. Witso, E., Persen, L., Benum, P., and Bergh, K. Cortical allograft as a vehicle for antibiotic delivery. Acta Orthop 76, 481, 2005.
11. Lord, C., Gebhardt, M., Tomford, W., and Mankin, H. Infection in bone allografts. Incidence, nature, and treatment. J Bone Joint Surg Am 70, 369, 1988.
12. Tomford, W., Thongphasuk, J., Mankin, H., and Ferraro, M. Frozen musculoskeletal allografts. A study of the clinical incidence and causes of infection associated with their use. J Bone Joint Surg Am 72, 1137, 1990.
13. Aro, H., and Aho, A. Clinical use of bone allografts. Ann Med 25, 403, 1993.
14. Gross, A., Hutchison, C., Alexeeff, M., Mahomed, N., Leitch, K., and Morsi, E. Proximal femoral allografts for reconstruction of bone stock in revision arthroplasty of the hip. Clin Orthop Relat Res 319, 151, 1995.
15. Bauer, T. Bone graft substitutes. Skelet Radiol 36, 1105, 2007.
16. Beardmore, A. Effectiveness of local antibiotic delivery with an osteoinductive and osteoconductive bone-graft substitute. J Bone Joint Surg Br 87, 107, 2005.
17. Winkler, H., Kaudela, K., Stoiber, A., and Menschik, F. Bone grafts impregnated with antibiotics as a tool for treating infected implants in orthopedic surgery-one stage revision results. Cell Tissue Bank 7, 319, 2006.
18. Jose, B., Antoci, V., Jr., Zeiger, A. R., Wickstrom, E., and Hickok, N.J. Vancomycin covalently bonded to titanium beads kills Staphylococcus aureus. Chem Biol 12, 1041, 2005.
19. Edupuganti, O. P., Antoci, V., Jr., King, S. B., Jose, B., Adams, C. S., Parvizi, J., Shapiro, I. M., Zeiger, A. R., Hickok, N.J., and Wickstrom, E. Covalent bonding of vancomycin to Ti6Al4V alloy pins provides long-term inhibition of Staphylococcus aureus colonization. Bioorg Med Chem Lett 17, 2692, 2007.
20. Antoci, V., Jr., Adams, C. S., Parvizi, J., Davidson, H. M., Composto, R. J., Freeman, T. A., Wickstrom, E., Ducheyne, P., Jungkind, D., Shapiro, I. M., and Hickok, N.J. The inhibition of Staphylococcus epidermidis biofilm formation by vancomycin-modified titanium alloy and implications for the treatment of periprosthetic infection. Biomaterials 29, 4684, 2008.
21. Antoci, V., Jr., King, S. B., Jose, B., Parvizi, J., Zeiger, A. R., Wickstrom, E., Freeman, T. A., Composto, R. J., Ducheyne, P., Shapiro, I. M., Hickok, N.J., and Adams, C. S. Vancomycin covalently bonded to titanium alloy prevents bacterial colonization. J Orthop Res 25, 858, 2007.
22. Antoci, V., Jr., Adams, C. S., Hickok, N.J., Shapiro, I. M., and Parvizi, J. Vancomycin bound to Ti rods reduces periprosthetic infection: preliminary study. Clin Orthop Relat Res 461, 88, 2007.
23. Peersman, G., Laskin, R., Davis, J., and Peterson, M. Infection in total knee replacement: a retrospective review of 6489 total knee replacements. Clin Orthop Relat Res 392, 15, 2001.
24. Loll, P. J., and Axelsen, P. H. The structural biology of molecular recognition by vancomycin. Ann Rev Biophys Biomol Struc 29, 265, 2000.
25. Anderson, H. Molecular biology of matrix vesicles. Clin Orthop Relat Res 314, 266, 1995.
26. Landis, W., and Silver, F. Mineral deposition in the extracellular matrices of vertebrate tissues: identification of possible apatite nucleation sites on type I collagen. Cells Tissues Organs 189, 20, 2009.
27. Fux, C., Costerton, J., Stewart, P., and Stoodley, P. Survival strategies of infectious biofilms. Trends Microbiol 13, 34, 2005.
28. Schopka, S., Schmid, F., Hirt, S., Birnbaum, D., Schmid, C., and Lehle, K. Recellularization of biological heart valves with human vascular cells: in vitro hemocompatibility assessment. J Biomed Mater Res Part B Appl Biomater 88, 130, 2009.
29. Rieder, E. Tissue engineering of heart valves: decellularized porcine and human valve scaffolds differ importantly in residual potential to attract monocytic cells. Circulation 111, 2792, 2005.
30. Ott, H., Matthiesen, T., Goh, S., Black, L., Kren, S., Netoff, T., and Taylor, D. Perfusion-decellularized matrix: using nature's platform to engineer a bioartificial heart. Nat Med 14, 213, 2008.
31. Ye, X., Zhao, Q., Sun, X., and Li, H. Enhancement of mesenchymal stem cell attachment to decellularized porcine aortic valve scaffold by in vitro coating with antibody against CD90: a preliminary study on antibody-modified tissue-engineered heart valve. Tissue Eng A 15, 1, 2008.
32. Antoci, V., Jr., King, S. B., Jose, B., Parvizi, J., Zeiger, A. R., Wickstrom, E., Freeman, T. A., Composto, R. J., Ducheyne, P., Shapiro, I. M., Hickok, N. J., and Adams, C. S. (2007) Journal of Orthopaedic. Research 25, 858-866.
33. Antoci, V., Jr., Adams, C. S., Parvizi, J., Ducheyne, P., Shapiro, I. M., and Hickok, N. J. (2007) Clin. Orthop. 461, 81-87.
34. Jose, B., Antoci, V., Jr., Zeiger, A. R., Wickstrom, E., and Hickok, N. J. (2005) Chemistry & Biology 12, 1041-1048
35. Holgers, K. M. and Ljungh, A. (1999) Biomaterials. 20, 1319-1326
36. Toljanic, J. A., Morello, J. A., Moran, W. J., Panje, W. R., and May, E. F. (1995) International Journal of Oral & Maxillofacial Implants 10, 578-582
37. Srinivasan, A., Dick, J. D., and Perl, T. M. (2002) Clinical Microbiology Reviews 15, 430 438
38. Walsh, T. R. and Howe, R. A. (2002) Annual Review of Microbiology 56, 657-675
39. Malabarba, A. and Ciabatti, R. (2001) Current Medicinal Chemistry 8, 1759-1773
40. Walsh, C. T. (2002) Chembiochem 3, 125-134
41. Loll, P. J. and Axelsen, P. H. (2000) Annual Review of Biophysics & Biomolecular Structure 29, 265-289
42. Jacobs, M. R., Jones, R. N., and Giordano, P. A. (2007) Diagnostic Microbiology & Infectious Disease 57, 55S-65S
43. Khawcharoenporn, T. and Alan, T. (2006) Hawaii Medical Journal 65, 290-293
44. Parvizi, J., Wickstrom, E., Zeiger, A. R., Adams, C. S., Shapiro, I. M., Purtill, J. J., Sharkey, P. F., Hozack, W. J., Rothman, R. H., and Hickok, N. J. (2004) Clinical Orthopaedics & Related Research 33-38
45. Teufel, L., Schuster, K. C., Merschak, P., Bechtold, T., and Redl, B. (2008) Journal of Molecular Microbiology and Biotechnology 14, 193-200
46. Zahm, A. M., Bucaro, M. A., Srinivas, V., Shapiro, I. M., and Adams, C. S. (2008) Bone 43, 25-31
47. Pomidor, M. M., Cimildoro, R., Lazatin, B., Zheng, P., Gurr, J. A., Leigh, I. M., Janne, O. A., Tuan, R. S., and Hickok, N. J. (1999) Mol. Biol. Cell 10, 4299-4310
48. Ottolenohi C E. Massive Osteo and Osteo-articular Bone Grafts Technic and Results of 62 Cases. Clin Orthop Relat Res. 1972; 87:156.
49. Shao Q D, Yan X, Sun J Y, Xu T M. Internal hemipelvectomy with reconstruction for primary pelvic neoplasm: a systematic review. ANZ J Surg. 2014:n/a-n/a. doi: 10.1111/ans. 12895.
50. Day R E, Megson S, Wood D. Iontophoresis as a means of delivering antibiotics into allograft bone. J Bone Joint Surg Br. 2005; 87(11):1568-1574. doi:10.1302/0301-620X.87B 11.16384.
51. Ketonis C, Barr S, Shapiro I M, Parvizi J, Adams C S, Hickok N J. Antibacterial activity of bone allografts: comparison of a new vancomycin-tethered allograft with allograft loaded with adsorbed vancomycin. Bone. 2011; 48(3):631-638. doi: 10.1016/j.bone.2010.10.171.
52. Ketonis C, Adams C S, and Barr S, et al. Antibiotic modification of native grafts: improving upon nature's scaffolds. Tissue engineering Part A. 2010; 16(6):2041-2049. doi:10.1089/ten.TEA.2009.0610.
53. Ketonis C, Barr S, Adams C S, Hickok N J, Parvizi J. Bacterial colonization of bone allografts: establishment and effects of antibiotics. Clin Orthop Relat Res. 2010; 468(8):2113-2121. doi: 10.1007/s11999-010-1322-8.
54. Ketonis C, Barr S, Adams C S, Shapiro I M, Parvizi J, Hickok N J. Vancomycin bonded to bone grafts prevents bacterial colonization. Antimicrobial Agents and Chemotherapy. 2011; 55(2):487-494. doi:10.1128/AAC.00741-10.
55. Lord C F, Gebhardt M C, Tomford W W, Mankin H J. Infection in bone allografts. Incidence, nature, and treatment. J Bone Joint Surg Am. 1988; 70(3):369-376.
56. Saegeman V, Lismont D, Verduyckt B, Ectors N, Stuyck J, Verhaegen J. Antimicrobial susceptibility of coagulase-negative staphylococci on tissue allografts and isolates from orthopedic patients. J Orthop Res. 2007; 25(4):501-507. doi: 10.1002/jor.20212.
57. Tomford W W, Thongphasuk J, Mankin H J, Ferraro M J. Frozen musculoskeletal allografts. A study of the clinical incidence and causes of infection associated with their use. J Bone Joint Surg Am. 1990; 72(8): 1137-1143.
58. Hicok K C, Thomas T, Gori F, Rickard D J, Spelsberg T C, Riggs B L. Development and characterization of conditionally immortalized osteoblast precursor cell lines from human bone marrow stroma. J Bone Miner Res. 1998; 13(2):205-217.
59. Rudert M, Holzapfel B M, Rottkay von E, Holzapfel D E, Noeth U. Impaction-Bone-Grafting zur Rekonstruktion ausgedehnter Knochendefekte beim Knieprothesenwechsel. Oper Orthop Traumatol. 2015; 27(1):35-46. doi: 10.1007/s00064-014-0330-3.
60. Salvati E A, Chekofsky K M, Brause B D, Wilson P D. Reimplantation in infection: a 12-year experience. Clin Orthop Relat Res. 1982; (170):62-75.
70. Delloye C, van Cauter M, Dufrane D, Francq B G, Docquier P-L, Cornu O. Local complications of massive bone allografts: an appraisal of their prevalence in 128 patients. Acta orthopaedica Belgica. 2014; 80(2): 196-204.
71. Atkinson, B. A (1986) Species incidence and trends of susceptibility to antibiotics in the United States and other countries: MIC & MBC. In: Antibiotics in laboratory medicine, ed. Lorain. V. The Williams & Wilkins Co. Baltimore, USA, pp. 995-1162.
72. Chopra. I. & Howe, T. G. B (1978) Bacterial resistance to the tetracyclines. Microbiology review 42, 707-724.
73. Ketonis C, Parvizi J, Adams C S, Shapiro I M, Hickok N J. Topographic features retained after antibiotic modification of Ti alloy surfaces: retention of topography with attachment of antibiotics. Clin Orthop Relat Res. 2009; 467(7):1678-1687. doi: 10.1007/s11999-009-0828-4.
74. Neut D, van de Belt H, Stokroos I, van Horn J R, van der Mei H C, Busscher H J. Biomaterial-associated infection of gentamicin-loaded PMMA beads in orthopaedic revision surgery. J Antimicrob Chemother. 2001; 47(6):885-891.
75. Costerton J W. Biofilm theory can guide the treatment of device-related orthopaedic infections. Clin Orthop Relat Res. 2005; (437):7-11.
76. Antoci V, Adams C S, Hickok N J, Shapiro I M, Parvizi J. Antibiotics for local delivery systems cause skeletal cell toxicity in vitro. Clin Orthop Relat Res. 2007; 462:200-206. doi:10.1097/BLO.0b013e31811ff866.
77. Gomes P S, Fernandes M H. Effect of therapeutic levels of doxycycline and minocycline in the proliferation and differentiation of human bone marrow osteoblastic cells. Arch Oral Biol. 2007; 52(3):251-259. doi:10.1016/j.archoralbio.2006.10.005.
78. Almazin S M, Dziak R, Andreana S, Ciancio S G. The effect of doxycycline hyclate, chlorhexidine gluconate, and minocycline hydrochloride on osteoblastic proliferation and differentiation in vitro. J Periodontol. 2009; 80(6):999-1005. doi: 10.1902/jop.2009.080574.

What is claimed is:

1. A method for manufacturing a bone allograft tissue comprising a covalently linked antibiotic comprising:
a. revealing primary amines through partial demineralization of the bone allograft;
b. coupling followed by deprotection of between one and four F-moc AEEA linkers using HATU chemistry to the primary amines;
c. coupling of antibiotic using either HATU chemistry of a carboxylic acid if available on the antibiotic or succinyl chemistry if only a primary amine or amide is available on the antibiotic; and
d. eluting of the unbound antibiotic leaving antibiotic covalently bound to tissue.

2. The method of claim 1 further comprising a first step of hydrating the bone before step a.

3. The method of claim 1 wherein the concentration of primary amines is at least 3.8×10⁻⁵ moles/mg bone.

4. The method of claim 1 wherein the ratio of primary amines to the number of coupled antibiotics is at least 6:1.

5. The method of claim 1 wherein the amount of antibiotic is at least 26 ng/mg of bone.

6. The method of claim 1, wherein the antibiotic is selected from the group consisting of peptide antibiotics, tetracycline antibiotics, and glycylcycline antibiotics, and combinations thereof.

7. The method of claim 6, wherein the antibiotic is selected from the group consisting of doxycycline, tetracycline, vancomycin, and combinations thereof.

8. A functionalized bone material comprising at least one linker covalently bonded to a primary amine group on the surface of the functionalized bone material, wherein a pharmaceutical composition is further bonded to the linker.

9. The functionalized bone material of claim 8, wherein the pharmaceutical composition is an antibiotic.

10. The functionalized bone material of claim 9 wherein the antibiotic is selected from the group consisting of peptide antibiotics, tetracycline antibiotics, and glycylcycline antibiotics, and combinations thereof.

11. The functionalized bone material of claim 8 wherein the concentration of primary amines is at least 3.8×10⁻⁵ moles/mg bone.

12. The functionalized bone material of claim 8 wherein the ratio of primary amines to the number of coupled antibiotics is at least 6:1.

13. The functionalized bone material of claim 8 wherein the amount of antibiotic is at least 26 ng/mg of bone.

14. A method for manufacturing a tissue comprising a covalently linked antibiotic comprising:
a. revealing a sufficient number of primary amines on the surface of the tissue;
b. coupling followed by deprotection of between one and four F-moc AEEA linkers using HATU chemistry to the primary amines;
c. coupling of a suitable antibiotic to said linkers; and
d. eluting of the unbound antibiotic leaving antibiotic covalently bound to the linker.

15. The method of claim 14 wherein the tissue is selected from the group consisting of bone allograft, bone autograft, venous grafts, cardiac grafts, tendons, ligaments, and combinations thereof.

16. The method of claim 14 wherein the sufficient number of primary amine are generated through partial demineralization of the tissue.

17. The method of claim 14 wherein the sufficient number of primary amine is 26 ng/mg of bone.

18. The method of claim 14 wherein the concentration of primary amines is at least 3.8×10⁻⁵ moles/mg bone.

19. The method of claim 14 wherein the ratio of primary amines to the number of coupled antibiotics is at least 6:1.

20. The method of claim 14, wherein coupling of the antibiotic is performed using HATU chemistry if a carboxylic acid is available on the antibiotic or succinyl chemistry if only a primary amine or amide is available on the antibiotic.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
